(19) United States
(12) Patent Application Publication
(10) Pub. No.: US 2018/0228721 A1
(43) Pub. Date: Aug. 16, 2018

(54) Functional and capable cosmetic compositions, that are appeasing to the senses, particularly visual and psyche, useful for social interaction, suitable for topical skin care application, and other applications, and methods of making same

(71) Applicant: JONAH LOVE, VALENCIA, CA (US)

(72) Inventor: JONAH LOVE, VALENCIA, CA (US)

(21) Appl. No.: 15/433,971

(22) Filed: Feb. 15, 2017

(51) Int. Cl. A61K 8/92; A61K 8/67; A61Q 19/00; B29C 39/00; B29C 39/10; B29C 39/38; B29C 63/04; B29C 69/00

(52) U.S. Cl. CPC A61K 8/927; A61K 8/922; A61K 8/678; A61Q 19/00; B29C 39/003; B29K 2091/00; B29C 39/38; B29C 63/04; B29C 69/001; A61K 2800/43; B29C 39/10

(57) ABSTRACT

A functional and capable cosmetic composition, that is appeasing to the senses, particularly visual and psyche, useful for social interaction, and topical skin care application. The composition primarily includes oil, butter, wax, and prong. The composition is solid at room temperature. A method of making the composition includes melting oil, butter, and wax, optional essential oil, vitamin E oil, and coloring, to form a liquid solution. Then the liquid solution is poured into a shaped mold and cooled to form a solid shaped component. The solid shaped component is then partially melted at exposed area to form a semi-solid, prong inserted into the exposed partially melted area of the component, and cooled to harden. Upon composition hardening, its removed from the mold to form the functional and capable cosmetic composition. Optional wrapping can be applied to and around composition to further appease the senses and usefulness for social interaction.

### BACKGROUND OF THE INVENTION

### 1. Field of the Invention

The present invention relates to cosmetic compositions, in particular to cosmetic compositions engineered to have function and capability, that appease the senses, particularly visual and psyche, which are primarily composed of oil, butter, wax, and prong, with optional essential oil, vitamin E oil, coloring, and wrapping, useful for topical skin care application, and are suitable for social interaction.

### 2. Background

Many cosmetic compositions are presented in containers or as plain blocks; such as creams in jars, lotion as bar, and serums in vials.

Several limitations exist as it pertains to the functionality and capability of cosmetic compositions which try to appease the senses, practically visual and psyche, as well as its usefulness for social interaction.

Cosmetic compositions which try to appease the visual and psychological sense may have the drawback of neither having the function of propping, nor the capability of producing varying presentations, nor use for social interaction. For example, lotion blocks, which try to appease the visual and psychological senses, do not have the functionality of propping into propping material, nor capability to produce variety of presentations such as floral bouquets.

Accordingly, there is a need for cosmetic compositions engineered to have function of propping and the capability to produce variety of presentations that are appeasing to the senses, particularly visual and psyche, useful for social interaction, and suitable for topical skin care application, and processes of preparing such compositions. The present application satisfies these and other needs, and provides further related advantages, as will be made apparent by the description of the embodiments herein the disclosure.

### BRIEF SUMMARY OF THE INVENTION

Presented herein are cosmetic compositions engineered to have function and capability, and processes of preparing such compositions. The compositions can appease the senses, particularly the visual and psyche, useful for social interaction, and suitable for topical skin care application. The compositions are solid at room temperature, at about 72 degree F. The compositions serve as an alternative to plain block and container held cosmetic compositions, for example.

In some embodiments, the functional and capable cosmetic composition is primarily composed of oil, butter, wax, and prong.

In some embodiments, the oil, butter, and wax mixture can further include, one or more of, essential oil, vitamin E oil, coloring, and mixtures thereof.

In some embodiments, the functional and capable composition can include optional wrapping.

In some embodiments, the oil can be almond, apricot kernel, argon, avocado, borage seed, camellia seed, cranberry seed, castor, coconut, babassu, black cumin seed, borage seed, flaxseed, grape seed, hazelnut, hemp seed, jojoba, kukui nut, macadamia nut, meadowfoam, olive, palm kernel, pomegranate seed, evening primrose, rosehip seed, safflower, shea, seabuckthorn, sesame, soybean, sunflower, tamanu, and mixtures thereof.

In some embodiments, the butter can be almond, aloe, apricot, avocado, chaulmoogra, cocoa, coconut, cupuacu, dhupu, hemp, lllipe, jojoba, kokum, kukui, macadamia, mango, mowrah, sal, shea, tucuma, ucuuba, and mixtures thereof.

In some embodiments, the wax can be bayberry, beeswax, candelilla, carnauba, castor, champaca, grapefruit, jasmine, jojoba, lanolin alcohol, microcrystalline, mimosa, orange peel, palm, sumac, sunflower, soy, rice bran, rose centifolia, tuberose, ozokerite, and mixtures thereof. In some embodiments, the essential oil can be allspice, anise star, basil, bay leaf, benzoin, bergamot, black pepper, cajeput, camphor, cardamom, carrot seed, cassia, cedarwood, chamomile, cinnamon, citronella, clary sage, clove, coriander, cypress, eucalyptus, fennel, fir needle, frankincense, geranium, ginger, grapefruit, helichrysum, jasmine, juniper, lavandin, lavender, lemon, lemongrass, lime, litsea cubeba, mandarin, marjoram, myrrh, neroli, niaouli, nutmeg, orange, oregano, palmarosa, patchouli, peppermint, petitgrain, pine needle, pomegranate, ravensara aromatic, rose, rosemary, sage, sandalwood, spearmint, spruce, tangerine, tea tree, thyme, vanilla, vetiver, wormwood, yarrow, ylang ylang, and mixtures thereof.

In some embodiments, the vitamin E oil can be natural, synthetic, and mixtures thereof.

In some embodiments, the coloring can be alfalfa leaf powder, beet juice, beet root powder, carrot juice extract, cabbage leaf extract, french clay, sweet potato powder, mica, red 5, turmeric root extract, and mixtures thereof.

In some embodiments, the functional and capable cosmetic composition can be molded into the shapes of, one or more of, being, object, imaginative being, imaginative object, communicative character, symbol, and mixtures thereof.

In some embodiments, the prong can be, one or more of, paper, plastic, wood, metal, stone, gem, glass, and mixtures thereof. In some embodiments, the type of prong used gives appeal to the cosmetic composition, appeasing the senses, particularly visual and psyche, as well as its use for social interaction.

In some embodiments, the cosmetic composition is wrapped, propped, and arranged to form variety of presentations which are appeasing to the senses, particularly visual and psyche, and provides usefulness for social interaction.

In some embodiments, the wrapping used to wrap the cosmetic composition are, one or more of, ribbon, cellophane, bow, tag, and mixtures thereof. In some embodiments, the cellophane wrapping preserves the composition and provides transparency which allows for appeal of the cosmetic composition. In some embodiments, ribbon is used to add color to the composition, adhere cellophane, create bows, and allows for placement of a tag.

In some embodiments, the ribbon and bow wrapping can be, one or more of, sheer, silk, cotton, satin, acetate, nylon, rayon, velvet, polyester, linen, hemp, ramie, jute, plastic, woven material, non-woven material, all-natural material, synthetic material, wired material, non-wired material, woven metals, aforementioned material printed with artistic designs, and mixtures thereof.

In some embodiments, the composition may be propped into propping material at different heights, angles, and placement locations. Additional wrapping can be placed between, below, and around propping material and propped composition to appease the senses, particularly visual and psyche, and provides usefulness for social interaction.

Also presented herein are processes for preparing functional and capable cosmetic compositions. In some embodiments, the process includes melting together mixture components of oil, a butter, and a wax to form a liquid solution, pouring liquid solution into shaped mold where it is cooled completely to room temperature to form shaped solid component, partially melting shaped solid component to a semi-solid at exposed surface, inserting prong to form pronged semi-solid component, cooling the pronged semi-solid component completely to room temperature to form a pronged solid composition, and removing composition from mold to form the functional and capable cosmetic composition.

In some embodiments, one or more of, essential oil, vitamin E oil, coloring, and mixtures thereof are added to the oil, butter, and wax mixture.

In some embodiments, functional and capable cosmetic composition is wrapped with wrapping. Also presented herein are processes for wrapping functional and capable cosmetic composition.

In some embodiments, the process includes adhering ribbon to prong immediately under shaped solid component, twirling ribbon around the prong until bottom of prong is reached, cutting off any excess ribbon which extends past the prong's end, and adhering twirled ribbon end to prong's end to form the functional and capable cosmetic.

### BRIEF DESCRIPTION OF THE DRAWINGS

The accompanying drawings, which are incorporated herein and forms a part of the specification, illustrates the present invention by way of example, and not by way of limitation. The drawings together with the description, further serves to explain the principle of the invention and to enable a person skilled in the pertinent art to make and use the invention.

FIG. 1 is a flowchart of an exemplary process for producing a functional and capable cosmetic composition, according to embodiments described herein.

FIG. 2 is a flowchart of an exemplary process for optional wrapping of a functional and capable cosmetic composition, according to embodiments described herein.

### DETAILED DESCRIPTION OF THE INVENTION

Unless defined otherwise, all technical and scientific terms used herein have the same meanings as commonly understood by one of ordinary skill in the art to which this invention belongs. Although any processes and materials similar or equivalent to those described herein can be used in the practice of the present invention, the preferred compositions, materials, and processes are described hereinafter. It will be apparent to a person skilled in the pertinent art that this invention can also be employed in a variety of other applications.

In order to further define this invention, the following terms and definitions are herein provided. As used herein when referring to any numerical value, the term “about” means a value of +/−10% of the stated value (e.g., “about 50 degree F.” encompasses a range of temperatures from 45 degree F. to 55 degree F., inclusive).

When the terms “one,” “a,” or “an” are used in the disclosure, they mean “at least one” or “one or more,” unless otherwise indicated. As such, the terms “a” (or “an”), “one or more,” and “at least one” can be used interchangeably herein.

As used herein, the term “function” or “functional” can be defined as the function of placement of a cosmetic composition into foam, vase, board, sand, and other propping materials; and in the context pertaining to topical skin care application, the ability to be leveraged for precision and controlled quantity application.

As used herein, the term “capable” or “capability” can be defined as the ability of a cosmetic composition being arranged, placed, and/or ordered into variety of presentations.

### Compositions

Presented herein are engineered functional and capable cosmetic compositions that primarily include an oil, butter, wax, and prong. The compositions are solid at room temperature, at about 72 degree F. In some embodiments, an exemplary functional and capable cosmetic composition include oil, butter, wax, essential oil, vitamin E oil, coloring, prong, and wrapping.

Within the context of the present invention, the composition is engineered to have function and capability, which will be understood by those of skill in the art, to mean that it has function to be placed into propping material as well as leveraged application of the composition when used as a topical skin care agent, and the capability to be arranged into varying placements, which are appeasing to the senses, particularly visual and psyche, and useful for social interaction.

The oil, butter, and wax mixture component of the compositions can provide shaping, securing, and skin benefits. The mixture can further include, one or more of, essential oil, vitamin E oil, coloring, and mixtures thereof. The mixture component can have a melting point of from about 111 degree F. to about 180 degree F. The melting point of the mixture component allows for shaping compositions and securing prong placement.

The oil component of the composition is a liquid. As used herein, the term “oil” does not include vitamin E oil or essential oils. Oils are extracted by physical or chemical extraction methods from one or more parts of plants. The wide range of vitamins and proteins, among other constituents in oils, help to heal skin, prevent damage to skin, and regenerate skin cells which are beneficial for skin and treatment of skin disease. The amount of the oil component of the mixture can be from about 20 wt % to about 55 wt %.

Examples of oils include almond, apricot kernel, argon, avocado, borage seed, camellia seed, cranberry seed, castor, coconut, babassu, black cumin seed, borage seed, flaxseed, grape seed, hazelnut, hemp seed, jojoba, kukui nut, macadamia nut, meadowfoam, olive, palm kernel, pomegranate seed, evening primrose, rosehip seed, safflower, shea, seabuckthorn, sesame, soybean, sunflower, and tamanu.

The butter component is a solid to soft-solid at room temperature, about 72 degree F. Butters are extracted by physical or chemical extraction methods from one or more parts of plants. Many butters have fatty acids and vitamins among other constituents which make butters a natural healing, restoring, and skin protectant. The amount of the butter component of the mixture can be from about 10 wt % to about 45 wt %.

Examples of butters include almond, aloe, apricot, avocado, chaulmoogra, cocoa, coconut, cupuacu, dhupu, hemp, lllipe, jojoba, kokum, kukui, macadamia, mango, mowrah, sal, shea, tucuma, and ucuuba.

The wax component of the composition can provide a crystalline structure allowing the composition to hold to the shapes of the molds to which the composition is molded to. The wax components are liquid-to-solid at room temperature, about 72 degree F. The components of many of these waxes include volatile oils, carotenoids, flavonoids, and aromatic plant acids, among other constituents, which give waxes its antiseptic, anti-inflammatory, healing, moisturizing, and soothing effect on skin. The amount of the wax component of the mixture can be from about 10 wt % to about 70 wt %.

Examples of waxes include bayberry, beeswax, candelilla, carnauba, castor, champaca, grapefruit, jasmine, jojoba, lanolin alcohol, microcrystalline, mimosa, orange peel, palm, sumac, sunflower, soy, rice bran, rose centifolia, tuberose, and ozokerite

In some embodiments, the composition can include an essential oil. As used herein, an “essential oil” is a concentrated, hydrophobic liquid containing volatile aroma compounds from plants. Essential oils are derived from various parts of plants. Essential oils have skin healing properties due to, its antibacterial, anti-inflammatory, antiviral, molecular size, and collagen enhancement properties. Essential oils are very effective for treating the skin and skin disease, which include, acne, chafing, dryness, fungal infections, microbial infections, and combinations thereof. An amount of the essential oil component of mixture can be from about 0.001 wt % to about 3 wt %. Examples of essential oils include allspice, anise star, basil, bay leaf, benzoin, bergamot, black pepper, cajeput, camphor, cardamom, carrot seed, cassia, cedarwood, chamomile, cinnamon, citronella, clary sage, clove, coriander, cypress, eucalyptus, fennel, fir needle, frankincense, geranium, ginger, grapefruit, helichrysum, jasmine, juniper, lavandin, lavender, lemon, lemongrass, lime, litsea cubeba, mandarin, marjoram, myrrh, neroli, niaouli, nutmeg, orange, oregano, palmarosa, patchouli, peppermint, petitgrain, pine needle, pomegranate, ravensara aromatic, rose, rosemary, sage, sandalwood, spearmint, spruce, tangerine, tea tree, thyme, vanilla, vetiver, wormwood, yarrow, and ylang ylang.

In some embodiments, the composition can include vitamin E oil. As used herein, the term “vitamin E oil” is also known as “tocopherol”. Vitamin E oil can be sourced from natural and synthetic sources. Due to vitamin E oil's antioxidant properties, vitamin E oil helps speed up cell regeneration, protects from free radicals, and supports new skin cell growth. An amount of the vitamin E oil component of the mixture can be from about 0.01 wt % to about 8 wt %.

In some embodiments, the composition can include coloring. The coloring can be sourced naturally or synthetically. Coloring can be soluble or insoluble. Coloring gives appeal to the composition, appeasing the senses, particularly the visual and psyche, and usefulness for social interaction. An amount of the coloring component of the mixture can be from about 0.0001 wt % to about 5 wt %.

Examples of coloring include alfalfa leaf powder, beet juice, beet root powder, carrot juice extract, cabbage leaf extract, french clay, mica, red 5, sweet potato powder, and turmeric root extract.

In some embodiments, the composition is shaped into the shape of, one or more of, being, object, imaginative being, imaginative object, communicative character, symbol, and mixtures thereof. The prong component of the composition adds function and capability by giving the composition propping ability into propping material and capability of arrangement. In some embodiments, the type of prong used gives appeal, appeasing the senses, particularly the visual and psyche, and usefulness for social interaction.

In some embodiments, the prong component can be, one or more of, paper, plastic, wood, metal, stone, gem, glass, and mixtures thereof.

In some embodiments, the composition can include wrapping, which is appeasing to the senses, particularly visual and psyche, and is useful for social interaction. In some embodiments, the type of wrapping used includes, one or more of, ribbon, cellophane, bow, tag, and mixtures thereof. In some embodiments, the wrapping provides preservation of the composition. In some embodiments, the ribbon can be, one or more of, sheer, silk, cotton, satin, acetate, nylon, rayon, velvet, polyester, linen, hemp, ramie, jute, plastic, woven material, non-woven material, all-natural material, synthetic material, wired material, non-wired material, woven metals, aforementioned material printed with artistic designs, and mixtures thereof. In some embodiments, the ribbon serves the purpose of securing the cellophane around the composition, creating bows, and placement of tags.

In some embodiments, the composition may be propped into propping material at different heights, angles, and placement locations. Additional wrapping can be placed between, below, and around propping material and propped compositions to appease the senses, particularly visual and psyche, and useful for social interaction.

In some embodiments, the functional and capable cosmetic composition is a topical skin care agent.

In some embodiments, the composition may be applied by leveraging prong to apply composition in the quantity desired with precision onto desired skin location, as a topical skin care agent. Such topical skin care agent treats the skin and skin disease, which include, dryness, chafing, acne, rashes, fungal infections, and microbial infections.

In other embodiments, further additional components suitable for inclusion in the compositions of the present invention will be familiar to the ordinarily skilled artisan.

Process of Preparing

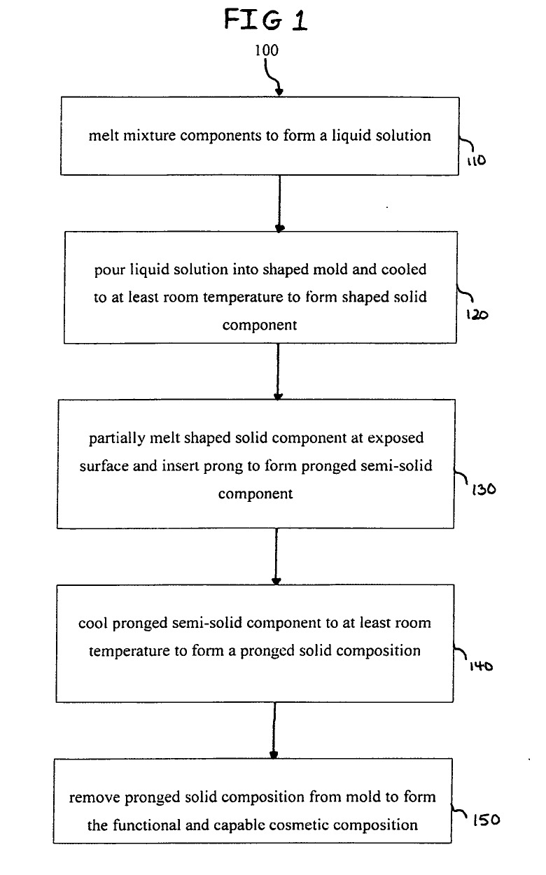

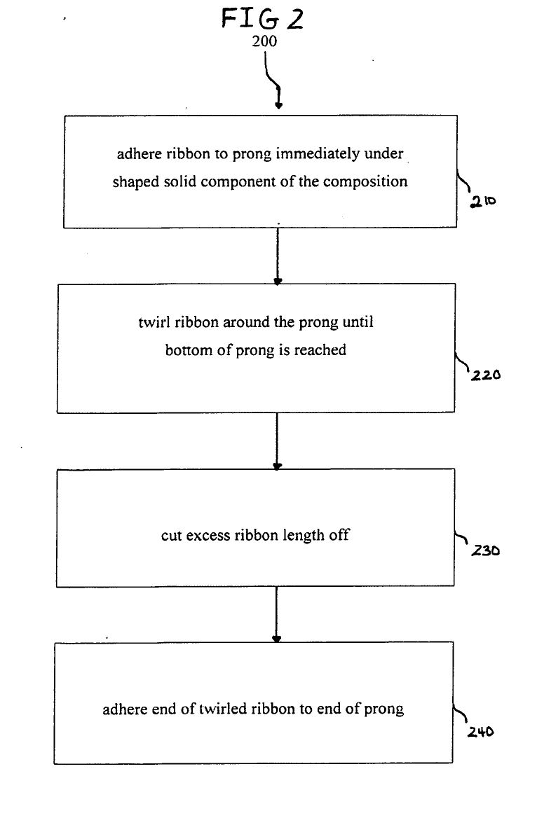

The processes for preparing functional and capable cosmetic compositions, including but not limited to the exemplary compositions described above, will now be described with reference to FIG. 1 and FIG. 2. As shown in FIG. 1, an exemplary process 100 for preparing functional and capable cosmetic composition, includes steps 110, 120, 130, 140, and 150. In step 110, mixture components oil, butter, and wax with optional essential oil, vitamin E oil, and coloring, are melted to form a liquid solution. The temperature for melting can be from about 111 degree F. to about 180 degree F. The oil, butter, and wax with optional essential oil, vitamin E oil, and coloring mixture component can be melted to form a liquid solution in a double boiler or a wax-melter, for example, or any other suitable appliance known in the art. In step 120 of exemplary process 100, the liquid solution is poured via wax-melter valve, for example, or any other suitable appliance know in the art, into one or more molds and transferred on a tray into a commercial refrigerator to be cooled completely from about 41 degree F. to about 72 degree F., upon hardening, removed from refrigerator to form shaped solid component. The molds are in the shape of, one or more of, being, object, imaginative being, imaginative object, communicative character, symbol, and mixtures thereof. In step 130, isolated heat is applied to the exposed surface of the shaped solid component until exposed shaped solid component's surface changes form to a semi-solid, at which point the prong is inserted to form a pronged semi-solid component. The isolated heat can be produced via a heat gun, for example, or any other suitable application known in the art. The temperature of the heat gun is at about 150 degrees F. The depth of the prong insertion is from about 25% to about 55% of the mold's cavity height. In step 140, the pronged semi-solid component is transferred into the commercial refrigerator at a temperature from about 41 degree F. to about 72 degree F. to be completely cooled, and upon hardening removed from the refrigerator to form a pronged solid composition. In step 150, solid composition is removed from the mold by gently peeling mold outward from the center of the composition's exposed surface while keeping prong securely intact, to form a functional and capable cosmetic composition.

As shown in FIG. 2, an exemplary process 200 for wrapping functional and capable cosmetic compositions, includes steps 210, 220, 230, and 240. In step 210, ribbon is adhered to the prong immediately under shaped solid component of the composition. This is done by using glue from a glue gun, for example, or any other suitable appliance known in the art. The temperature of the glue gun is at about 190 degree F. Glue is applied onto prong, then the wrapping is applied to the glue and prong, the glue is allowed to cool for five seconds while glue, prong, and wrapping are intact together. In step 220, ribbon is twirled around the prong until bottom of prong is reached. In step 230, any excess ribbon length is cut using scissors, for example, or any other suitable tool known in the art. In step 240, twirled ribbon end is adhered to the end of the prong, with glue via glue gun, for example, or any other known appliance in the art. The temperature of the glue gun is at about 190 degree F. While prong, glue, and wrapping are adhered together, glue is allowed to cool for five seconds, forming the functional and capable cosmetic composition.

Use

The functional and capable cosmetic compositions are applied by leveraging prong to apply shaped component of composition in the quantity desired with precision onto desired skin location, as a topical skin care agent, for example. Such topical skin care treats the skin and skin disease.

Examples of skin treatment and skin disease are, dryness, chafing, acne, rashes, fungal infections, and microbial infections.

The functional and capable cosmetic compositions are useful for social interactions, such as expression of thanks, celebration, and love when given from one person or party to another, for example.

Example use of functional and capable cosmetic compositions in social interaction are, a flower shaped functional and capable cosmetic composition given on Valentine's Day from a husband to a wife, an arrangement of multiple flower and leaf shaped functional and capable cosmetic compositions in a basket as a gift of thanks given to an appreciated teacher, and a graduating-bear shaped functional and capable cosmetic composition given at a graduation party to the graduate.

The functional and capable cosmetic composition appeases the senses, particularly visual and psyche, such as when used as décor in homes, offices, and hospitals, for example.

Example use of functional and capable cosmetic compositions as décor are, animal, plant, and sun shaped and arranged functional and capable cosmetic compositions inserted into a foam board, depicting a jungle, placed in an office with a jungle theme setting. An orchid shaped functional and capable cosmetic composition given to a person in a hospital to comfort.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following example, which is included herewith for purposes of illustration only and is not intended to be limiting of the invention.

### Example

This Example describes an exemplary process for producing functional and capable cosmetic composition, including oils, butter, wax, essential oil, vitamin E oil, prong, and wrapping. The composition is prepared as follows: sunflower oil (35% wt), shea butter (35% wt), beeswax (20% wt), grape seed oil (8% wt), lavender essential oil (1% wt), and natural vitamin E oil (1% wt) are melted in a 45 gallon commercial grade wax-melter at about 170 degree F. The batch weighs about 245 pounds with a volume of about 30 gallons. The liquid solution is blended by stirring to ensure substantial uniform distribution of the ingredients. The mixture is then poured via the wax-melter valve into flower shaped molds having the approximate cavity diameter of 1.5 inch and cavity height of 1 inch.

The flower molds are then placed on a tray and transferred into a commercial refrigerator and left in refrigerator for about ten minutes where the liquid solution will harden to the shape of the flower molds. The refrigerator is at about 41 degree F.

Upon hardening of the liquid solution in the molds, the molds are taken out of the refrigerator and isolated heat is applied to the exposed surface of each exposed hardened flower shaped component using a heat gun set to about 150 degree F. Isolated heat is applied at about 6 inches from the surface of each, exposed, hardened shaped component. Upon the exposed area of the hardened shaped component changing form to a semi-solid, paper prong is inserted about 0.25 inches deep vertically into the center of the exposed, semi-solid, surface of the component. Prong placement is repeated to each molded component. The prongs are six inches long and 0.25 inches in diameter. The molded components, with prongs inserted, is transferred on a tray into a commercial refrigerator and left in refrigerator to harden for about five minutes at a temperature at about 41 degree F.

Upon hardening, the tray is removed from the refrigerator, and solid flower shaped compositions are removed from molds by gently peeling molds outward from the center of each exposed surface of each solid flower shaped composition until solid flower shaped compositions are completely removed keeping prongs securely intact in compositions.

Each prong is wrapped with light green ribbon made of satin sheer using a glue gun. The glue gun has a temperature of about 190 degree F. The ribbon has a width of 0.75 inch and length of 12 inches. Glue is adhered to the prong immediately below flower shaped component, and the ribbon's shortest side, the width of the ribbon, is applied at about a 45 degree angle onto glue and prong while glue is still hot. Glue is allowed to dry for about five seconds while prong, glue, and wrapping are still adhered together. The ribbon is then twirled around the prong at a continued 45 degree angle covering all exposed surfaces of the paper prong, upon reaching the end of the prong, excess ribbon length is cut with scissor. The end of the twirled ribbon is glued to the end of the prong, using glue from glue gun, allowed to cool for about five seconds while glue, prong, and wrapping are still adhered together, and then placed on a cookie stick holder.

Cellophane is cut to about a 5-inch by 5-inch square and placed over the flower shaped component of the composition so that all cellophane tips meet on prong. Red ribbon made of woven polyester, 0.25 inch wide by 15 inch long, is used to adhere, by tying, all upper portion of cellophane's tips securely against the prong, knotted, and used to tie a bow. Tag of 0.75 inch high by 2.5 inch wide, with 0.25 inch diameter hole at middle of left side, located 0.25 inch inward from edge, is used to insert an end of the red ribbon through, glue adhered to the back of the tag where ribbon and tag meet, and allowed to cool for about five seconds while ribbon, tag, and glue are still adhered together.

The resulting composition is a functional and capable cosmetic composition solid at room temperature, having function to be propped and the capability to produce variety of presentations, which are appeasing to the senses, particularly visual and psyche, useful for social interaction, and suitable for topical skin care application.

All of the various embodiments and options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the claims and their equivalents.

What is claimed is:

1. A process for the preparation of a functional and capable cosmetic composition, comprising: i. melting together mixture components (a) an oil, (b) a butter, and (c) a wax to form a liquid solution; ii. pouring liquid solution into shaped mold where it is cooled completely to at least room temperature to form shaped solid component; iii. partially melting shaped solid component at exposed surface and inserting prong to form pronged semi-solid component; iv. cooling the pronged semi-solid component completely to at least room temperature to form a pronged solid composition; and v. removing composition from mold to form the functional and capable cosmetic composition.

2. The process according to claim 1, wherein the oil comprises from about 20 wt % to about 55 wt %, wherein the butter comprises from about 10 wt % to about 45 wt %, and wherein the wax comprises from about 10 wt % to about 70 wt % of the mixture components of the composition.

3. The process according to claim 1, wherein (a) the oil is sunflower oil, (b) the butter is shea butter, and (c) the wax is beeswax.

4. The process according to claim 1, wherein (a) the oil is selected from the group consisting of almond, apricot kernel, argon, avocado, borage seed, camellia seed, cranberry seed, castor, coconut, babassu, black cumin seed, borage seed, flaxseed, grape seed, hazelnut, hemp seed, jojoba, kukui nut, macadamia nut, meadowfoam, olive, palm kernel, pomegranate seed, evening primrose, rosehip seed, safflower, shea, seabuckthorn, sesame, soybean, sunflower, tamanu, and mixtures thereof.

5. The process according to claim 1, wherein (b) the butter is selected from the group consisting of almond, aloe, apricot, avocado, chaulmoogra, cocoa, coconut, cupuacu, dhupu, hemp, lllipe, jojoba, kokum, kukui, macadamia, mango, mowrah, sal, shea, tucuma, ucuuba, and mixtures thereof.

6. The process according to claim 1, wherein (c) the wax is selected from the group consisting of bayberry, beeswax, candelilla, carnauba, castor, champaca, grapefruit, jasmine, jojoba, lanolin alcohol, microcrystalline, mimosa, orange peel, palm, sumac, sunflower, soy, rice bran, rose centifolia, tuberose, ozokerite, and mixtures thereof.

7. The process according to claim 1, wherein (i) melting together mixture components comprises melting (a), (b), and (c) at a temperature of from about 111 degree F. to about 180 degree F.

8. The process according to claim 1, wherein (i) melting together mixture components comprises melting (a), (b), and (c) together with (d) from about 0.001 wt % to about 3 wt % of an essential oil.

9. The process according to claim 8, wherein (d) the essential oil is selected from the group consisting of allspice, anise star, basil, bay leaf, benzoin, bergamot, black pepper, cajeput, camphor, cardamom, carrot seed, cassia, cedarwood, chamomile, cinnamon, citronella, clary sage, clove, coriander, cypress, eucalyptus, fennel, fir needle, frankincense, geranium, ginger, grapefruit, helichrysum, jasmine, juniper, lavandin, lavender, lemon, lemongrass, lime, litsea cubeba, mandarin, marjoram, myrrh, neroli, niaouli, nutmeg, orange, oregano, palmarosa, patchouli, peppermint, petitgrain, pine needle, pomegranate, ravensara aromatic, rose, rosemary, sage, sandalwood, spearmint, spruce, tangerine, tea tree, thyme, vanilla, vetiver, wormwood, yarrow, ylang ylang, and mixtures thereof.

10. The process according to claim 8, wherein (i) melting together mixture components comprises melting (a), (b), (c), and (d) together with (e) from about 0.01 wt % to about 8 wt % of vitamin E oil.

11. The process according to claim 10, wherein (e) the vitamin E oil is selected from the group consisting of natural vitamin E oil, synthetic vitamin E oil, and mixtures thereof.

12. The process according to claim 10, wherein (i) melting together mixture components comprises melting (a), (b), (c), (d), and (e), together with (f) from about 0.0001 wt % to about 5 wt % of coloring.

13. The process according to claim 12, wherein (f) the coloring is selected from the group consisting of alfalfa leaf powder, beet juice, beet root powder, carrot juice extract, cabbage leaf extract, french clay, sweet potato powder, mica, red 5, turmeric root extract, and mixtures thereof.

14. The process according to claim 12, wherein (i) melting together mixture components comprises melting (a), (b), (c), (d), (e), and (f) at a temperature of from about 111 degree F. to about 180 degree F.

15. The process according to claim 1, wherein (i) melting together mixture components allows for shaping of the composition, prong placement, and uniform distribution of mixture components.

16. The process according to claim 1, wherein (ii) the mold is selected from a group consisting of (i) being, (ii) object, (iii) imaginative being, (iv) imaginative object, (v) communicative character, (vi) symbol, and mixtures thereof.

17. The process according to claim 1, wherein (ii) the poured liquid solution is hardened at a temperature of from about 41 degree F. to about 72 degree F.

18. The process according to claim 1, wherein (iii) the shaped solid component is partially melted by applying heat at its exposed surface while in mold at a temperature of about 150 degree F., allowing the insertion of a prong.

19. The process according to claim 1, wherein (iii) the prong is selected from a group consisting of (i) paper, (ii) plastic, (iii) wood, (iv) metal, (v) stone, (vi) gem, (vii) glass, and mixtures thereof.

20. The process according to claim 1, wherein (iv) the partially melted component with prong inserted is cooled to harden at a temperature of from about 41 degree F. to about 72 degree F.

21. The process according to claim 1, wherein (v) the composition is removed from the mold with prong securely intact in composition.

22. The process according to claim 1, wherein the functional and capable cosmetic composition is solid at room temperature, at about 72 degree F.

23. The process according to claim 1, wherein the functional and capable cosmetic composition is suitable for topical skin care application to treat skin and skin disease.

24. The process according to claim 1, wherein the functional and capable cosmetic composition provides for leveraged pressure, precision application, and quantity control when applied topically.

25. The process according to claim 1, wherein the functional and capable cosmetic composition is appeasing to the senses, particularly visual and psyche, and usefulness for social interaction.

26. The process according to claim 1, wherein the functional and capable cosmetic composition has the ability to be propped into propping material at different heights, angles, and placement locations, appeasing to the senses, particularly visual and psyche, and usefulness for social interaction.

27. A composition prepared by the process of claim 1.

28. A process for the preparation of a functional and capable cosmetic composition, consisting essentially of: i. melting together mixture components (a) an oil, (b) a butter, (c) a wax, optionally with one or more of, (d) an essential oil, (e) vitamin E oil, and (f) a coloring, to form a liquid solution; ii. pouring liquid solution into shaped mold where it is cooled completely to at least room temperature to form shaped solid component; iii. partially melting shaped solid component at exposed surface and inserting prong to form pronged semi-solid component; iv. cooling the pronged semi-solid component completely to at least room temperature to form a pronged solid composition; and v. removing composition from mold (1) to form functional and capable cosmetic composition, optionally, (2) composition is wrapped in one or more of the following ways, (a) directly onto the composition, (b) around the composition, and (c) onto propping material which props functional and capable cosmetic composition, to form functional and capable cosmetic composition.

29. The process according to claim 28, wherein (2) wrapping is selected from a group consisting of (a) ribbon, (b) cellophane, (c) bow, (d) tag, and mixtures thereof.

30. The process according to claim 28, wherein (2) (a) a process of wrapping directly onto the composition comprises: (i) adhering ribbon to prong immediately under shaped solid component, (ii) twirling ribbon around the prong until bottom of prong is reached, (iii) cutting off any excess ribbon which extends past the prong's end, and (iv) adhering twirled end of ribbon to prong's end.

31. The process according to claim 28, wherein the functional and capable cosmetic composition is appeasing to the senses, particularly visual and psyche, and useful for social interaction.

32. A composition prepared by the process of claim 28.
